(19) United States
(12) Patent Application Publication
(10) Pub. No.: US 2005/0124930 A1
(43) Pub. Date: Jun. 9, 2005

(54) Separable auxiliary device of milk absorber

(76) Inventor: Mu Hsiang Lin, Yi Lan Hsien (TW)

Correspondence Address: Mu Hsiang Lin, 235 Chung - Ho, Box 8-24, Taipei, TW

(21) Appl. No.: 10/726,959

(22) Filed: Dec. 4, 2003

(51) Int. Cl. A61M 1/06

(52) U.S. Cl. 604/74

(57) ABSTRACT

A separable auxiliary device of a milk absorber serves for sucking milk from a woman breast. An adjusting unit has a casing. An interior of the casing has a tube and a receiving space communicated to the tube. The receiving space is sequentially arranged with an elastic element, a plug and a drain-proof element. A cover is arranged on the drain-proof element. An interior of the cover has an adjusting hole and an operation hole which are communicated to the receiving space and external space. The operation hole is arranged with a rod portion. A key is installed on the rod portion. A transmission tube is connected to the tube. A suction unit is connected to another end of the tube. Thus, the auxiliary device is separable from the milk absorber for storage and transfer and the suction force is adjustable so that the user will feel easy.

### FIELD OF THE INVENTION

The present invention relates to milk absorbers, and particular to a separable auxiliary device of a milk absorber, in that the auxiliary device is separable from the milk absorber for storage and carry and the suction force is adjustable so that the user will feel easy.

### BACKGROUND OF THE INVENTION

Currently, milk absorber is used to suck milk from a woman breast so as to collect milk for feeding child. In the prior art, the milk absorber has a cover which is combined with a container. The cover has a cup one end of which covers the breast of a woman. Another end of the cup is connected to an outlet of the milk absorber for exhausting air. The outlet is connected with an air bag. When the cup covers the breast, the air bag is compressed so as to generate a suction force to suck the milk from the breast. The sucking milk will flow into the container. This milk absorber causes the woman to press the air bag using by hand, but the operation needs a great force and a longer time is necessary. The air bag is directly connected to the milk absorber and is inseparable. Thereby, it cannot be carried easily.

In another prior art, a pump is directly installed to a cover. When the pump is actuated, a cup will generate a suction force to suck breast milk. Moreover, the suction force cannot be adjusted easily so that the woman will feel uneasy. The pump is installed on the cover of the milk absorber. This not only increases the weight of the milk absorber, but also it causes an inconvenience in the suction process.

### SUMMARY OF THE INVENTION

Accordingly, the primary object of the present invention is to provide a separable auxiliary device of a milk absorber, in that the auxiliary device is separable from the milk absorber for storage and transfer and the suction force is adjustable so that the user will feel easy.

To achieve above objects, the present invention provides a separable auxiliary device of a milk absorber for sucking milk from a woman breast. An adjusting unit has a casing. An interior of the casing has a tube and a receiving space communicated to the tube. The receiving space is sequentially arranged with an elastic element, a plug and a drain-proof element. A cover is arranged on the drain-proof element. An interior of the cover has an adjusting hole and an operation hole which are communicated to the receiving space and external space. The operation hole is arranged with a rod portion. A key is installed on the rod portion. A transmission tube is connected to the tube. A suction unit is connected to another end of the tube. Thus, the auxiliary device is separable from the milk absorber for storage and transfer and the suction force is adjustable so that the user will feel easy.

The various objects and advantages of the present invention will be more readily understood from the following detailed description when read in conjunction with the appended drawing.

### BRIEF DESCRIPTION OF THE DRAWINGS

FIG. 1 is a schematic view showing the arrangement of the present invention.

FIG. 2 is an exploded schematic view of the adjusting unit of the present invention.

FIG. 3 is a lateral schematic view of FIG. 2 according to the present invention.

FIG. 4 is a schematic view showing the operation of the pressing of the adjusting unit of FIG. 3 according to the present invention.

FIG. 5 is a schematic view showing the suction unit of the present invention.

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

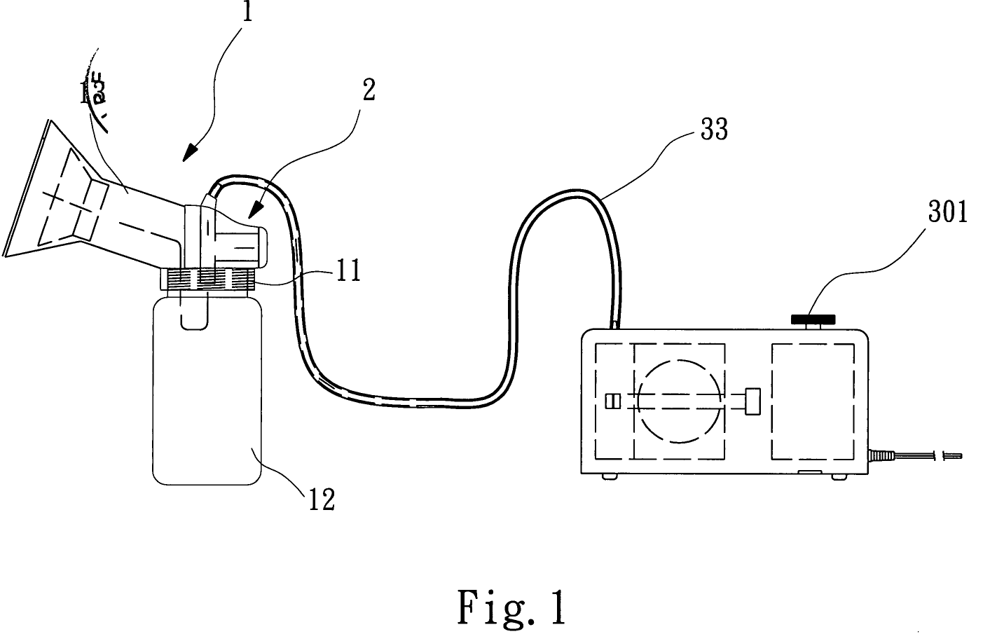

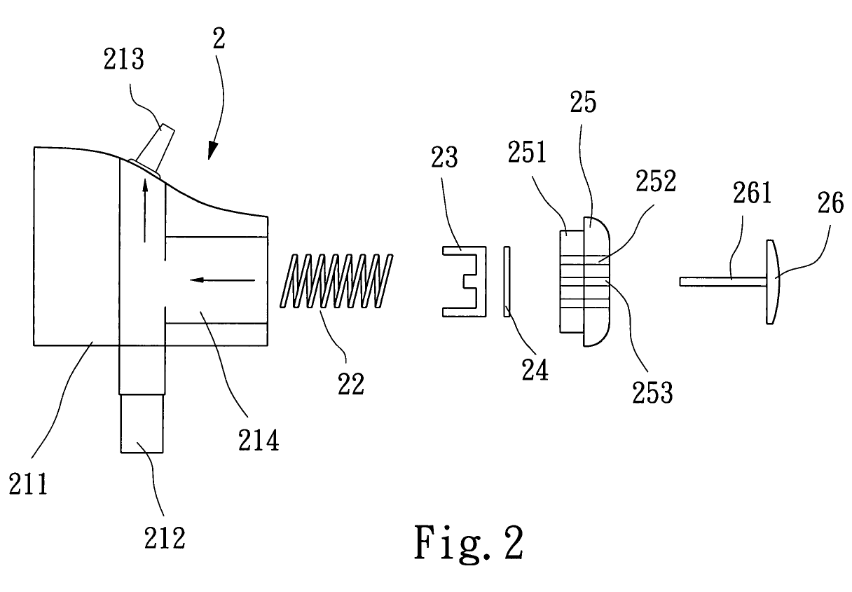

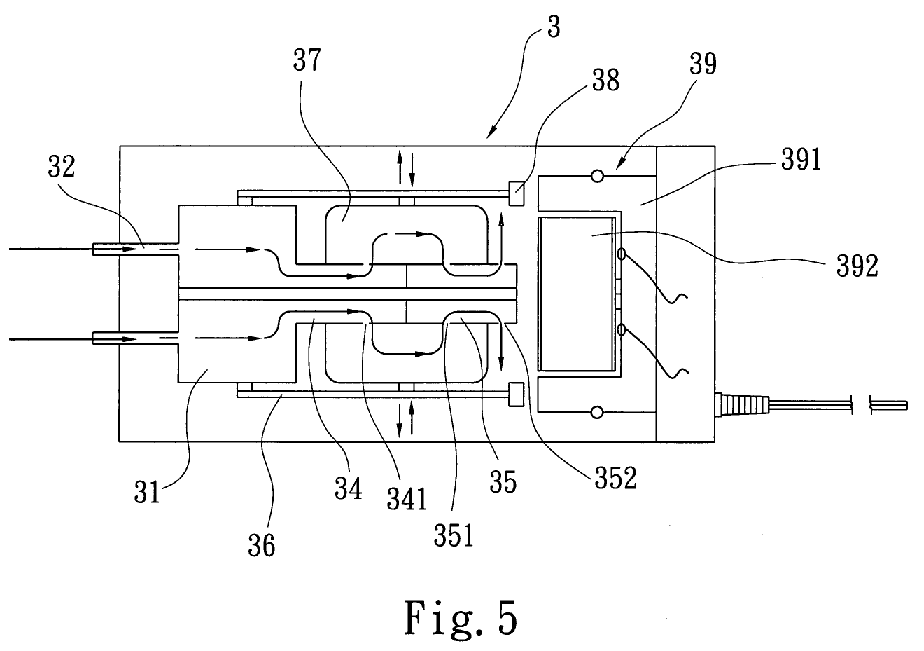

With reference to FIGS. 1, 2 and 5, the separable auxiliary device of a milk absorber of the present invention is illustrated. The separable auxiliary device of a milk absorber includes a milk absorbing unit 1, an adjusting unit 2 on the milk absorbing unit 1, a transmission tube 33 connected to the adjusting unit 2; and an suction unit 3 connected to the transmission tube 33.

In use, the milk absorbing unit 1 covers a breast of a woman. Then the suction unit 3 is actuated for sucking milk from the breast. In the sucking process, if the breast feels uneasy, the adjusting unit 2 is used to adjust the suction force or an adjusting button 301 on the suction unit 3 is used to adjust the suction force so that the user may feel easy. Thereby, the milk absorbing unit can be used to breasts of various sizes.

The above mentioned milk absorbing unit 1 has a cover 11. The cover 11 is combined with a container 12 for collecting milk. The cover 11 includes a cup 13 which can cover the breast of a woman.

The adjusting unit 2 is arranged on the cover 11. The adjusting unit 2 has a casing 21. A tube 211 is in the casing 21. One end of the tube 211 has an inlet 212. Another end thereof has an outlet 213 which is connected to the transmission tube 33. An interior of the casing 21 has a receiving space 214 which is communicated to the tube 211. An interior of the receiving space 214 is arranged with an elastic element 22 which is selectable from one of a spring, a reed and a rubber. The elastic element 22 has a plug 23. A drain-proof element 24 made of rubber is arranged on the plug 23. A cap 25 is arranged on the drain-proof element 24. A combining portion 251 is arranged on the cap 25, which is communicated to the receiving space 214. The cap 25 is formed with an adjusting hole 252 and an operation hole 253 which are communicated to the receiving space 214 and external space. A rod portion 261 is arranged into the operation hole 253. A key 26 is arranged on the rod portion 261.

An internal of the suction unit 3 has an air chamber 31. One end of the air chamber 31 is formed with at least one inlet 32 which is connected the transmission tube 33 and another end thereof has a first channel 34 which is communicated to the air chamber 31. A lateral side of the first channel has an outlet 341. One end of the first channel 34 is connected to a second channel 35. The first channel 34 is not communicated to the second channel 35. The second channel has an inlet 351 and an exhausting hole 352. At least one rod body 36 is connected at an outer side of the air chamber 31 at one end thereof. In this embodiment, two bodies 36 are illustrated. An air bag chamber 37 made of rubber is installed at a predetermined position of the rod body 36. The air bag chamber 37 is connected between the outlet 341 of the first channel 34 and the inlet 212 of the second channel 35. Another end of each rod body 36 has a driven element 38 made of magnet. A driving element 39 is installed in the suction unit 3 at a position corresponding to the driven element 38. The driving element 39 is formed by a silicon steel sheet 391 and a coil 392 for generating magnetic field to drive the driven element 38 to further drive the rod body 36 to move. Thus, the air bag chamber 37 is compressed between the outlet 341 of the first channel 34 and the inlet 212 of the second channel 35. Thus, air can flow into the inlet 32 of the air chamber 31 for sucking milk. Furthermore, the suction unit 3 has an adjusting button 301 for adjusting the current of the internal circuit of the suction unit 3 so as to adjust the sucking force of the suction unit 3. Thereby, women of various sizes of breasts can use the present invention and the breast milk can be sucked at a short time period.

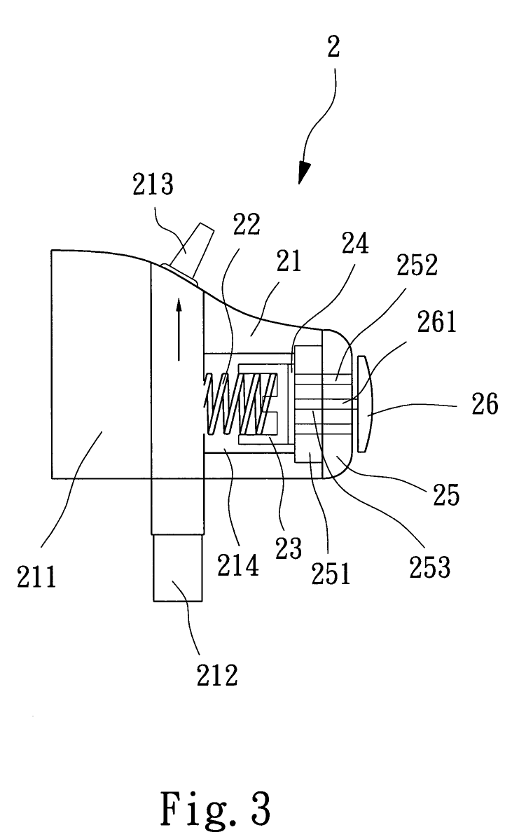

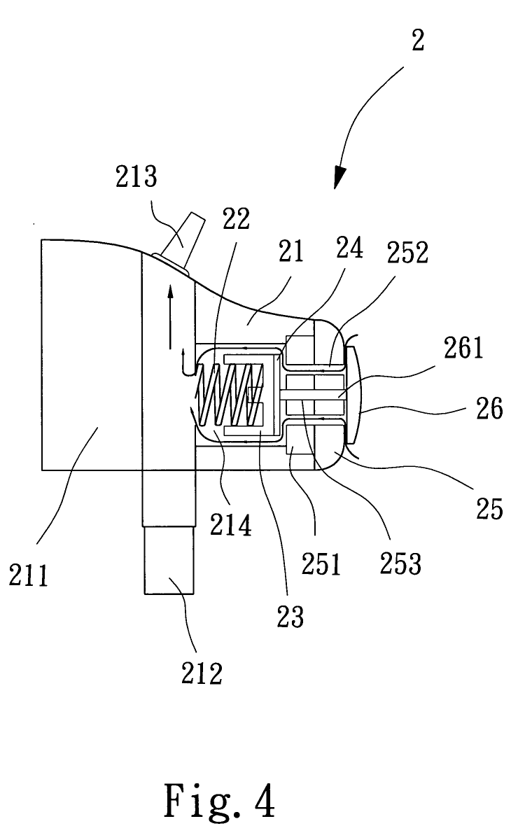

With reference to FIGS. 1, 3 and 4, the operation of the present invention is illustrated. In use, one end of the transmission tube 33 is connected to the outlet 213 of the adjusting unit 2 and another end of the transmission tube 33 is connected to the inlet 32 of the suction unit 3. Then the cup 13 of the milk absorbing unit 1 covers the breast of a woman. When the suction unit 3 is actuated, the milk flows into the container 12 of the milk absorbing unit 1 and air flows into the inlet 212 of the tube 211 of the adjusting unit 2. Then air flow through the transmission tube 33 to the interior of the suction unit 3.

If in the sucking process, the woman feels uneasy due to a too large sucking force. The woman can press the key 26 of the adjusting unit 2 so that the rod portion 261 pushes away the plug 23. External air flows into the receiving space 214 of the adjusting hole 252 of the cap 25 so as to reduce the sucking force between the breast and the cup 13. Thereby, in the sucking process, the women will not feel uneasy.

If the breast is too large or too small, or the milk is too much, the adjusting button 301 of the suction unit 3 can be rotated for adjusting the sucking force of the suction unit 3. Thereby, the milk can be sucked out in a short time period.

Moreover, when the milk absorbing unit 1 is not used, the transmission tube 33 is separated from the adjusting unit 2 and the suction unit 3 for storage and transfer.

Furthermore, a plurality of inlets 32 can be formed on the suction unit 3 so that the separable auxiliary device for a milk absorber is used to suck milk from two breasts at the same time. Or two milk absorbing units 1 are connected together to be used for two women.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A separable auxiliary device of a milk absorber for sucking milk from a woman breast; comprising:
an adjusting unit having a casing; an interior of the casing having a tube and a receiving space communicated to the tube; the receiving space being sequentially arranged with an elastic element, a plug and a drain-proof element; a cover being arranged on the drain-proof element; an interior of the cover having an adjusting hole and an operation hole which are communicated to the receiving space and external space; the operation hole being arranged with a rod portion, a key being installed on the rod portion;
a transmission tube connected to one end of the tube;
a suction unit connected to another end of the tube; the suction unit having at least one inlet which is connected to the transmission tube;
wherein the cover is combined with a container for collecting milk; the cover includes a cup for covering a breast of a user; when the suction unit is actuated, milk of the user will be sucked to the container; in the sucking process, if the user feels uneasy, the adjusting unit is pressed so as to reduce the suction force between the breast and the milk absorber so that the user will not feel uneasy in the suction process.

2. The separable auxiliary device of a milk absorber as claimed in claim 1, wherein one end of the tube has an inlet and another end of the tube has an outlet.

3. The separable auxiliary device of a milk absorber as claimed in claim 1, wherein the cover has a combining portion connected to the receiving space.

4. The separable auxiliary device of a milk absorber as claimed in claim 1, wherein the elastic element is formed by one of springs, reeds, and rubbers.

5. The separable auxiliary device of a milk absorber as claimed in claim 1, wherein the drain-proof element is made of rubber.

6. The separable auxiliary device of a milk absorber as claimed in claim 1, wherein an internal of the suction unit has an air chamber; one end of the air chamber is formed with at least one inlet which is connected the transmission tube and another end thereof has a first channel which is communicated to the air chamber; a lateral side of the first channel has an outlet; one end of the first channel is connected to a second channel; the first channel is not communicated to the second channel; the second channel has an inlet and an exhausting hole.

7. The separable auxiliary device of a milk absorber as claimed in claim 6, wherein at least one rod body is connected at an outer side of the air chamber at one end thereof; an air bag chamber is installed at a predetermined position of the rod body; the air bag chamber is connected between the outlet of the first channel and the inlet of the second channel; another end of each rod body has a driven element; a driving element is installed in the suction unit at a position corresponding to the driven element.

8. The separable auxiliary device of a milk absorber as claimed in claim 7, wherein the drain-proof element is made of magnet.

9. The separable auxiliary device of a milk absorber as claimed in claim 7, wherein the driving element is formed by a silicon steel sheet and a coil.

10. The separable auxiliary device of a milk absorber as claimed in claim 1, wherein the suction unit has an adjusting button for adjusting a current in an internal circuit of the suction unit so as to change a suction force of the suction unit.
